(19) United States
(12) Patent Application Publication
(10) Pub. No.: US 2002/0052660 A1
(43) Pub. Date: May 2, 2002

(54) Leak and tear resistant grafts

(76) Inventor: E. Skott Greenhalgh, Wyndmoor, PA (US)

Correspondence Address: John A. Chionchio, Esquire, Synnestvedt & Lechner LLP, 1101 Market Street, Suite 2600, Philadelphia, PA, 19107-2950, US

(21) Appl. No.: 09/998,878

(22) Filed: Oct. 31, 2001

Related U.S. Application Data
(60) Provisional application No. 60/244,490, filed on Oct. 31, 2000.

(51) Int. Cl. A61F 2/06; A61F 2/04; A61F 2/02

(52) U.S. Cl. 623/23.7; 623/23.74; 623/1.36; 623/1.54

(57) ABSTRACT

Leak and tear resistant grafts for repair and replacement of living animal tissue are disclosed. The grafts, which may be flat or tubular, are formed of interlaced filamentary members and have reinforced attachment regions defined by interlaced filamentary members of higher tensile strength. Higher strength is provided by use of high strength material, increased denier or number of plies of the filamentary members. High strength members inhibit propagation of tears which occur in the graft when the attachment region is pierced by sutures or staples. Filamentary members formed of textured, elastic or heat shrinkable yarns interlaced in the graft inhibit leakage.

### RELATED APPLICATION

This application is based on and claims priority of U.S. Provisional Application No. 60/244,490, filed Oct. 31, 2000.

### FIELD OF THE INVENTION

This invention concerns grafts having improved strength, fatigue, abrasion resistance and leak resistance characteristics and especially to grafts for use in human implants.

### BACKGROUND OF THE INVENTION

Woven, knitted and braided fabric structures are used extensively as grafts for human implants to repair vascular disorders such as aneurysms, coronary bypasses and the connection for the anastomosis of blood vessels or intestinal segments or to repair an abdominal wall such as in a hernia operation, to cite a few examples.

When used as an implant, the graft must be somehow attached to living animal tissue, for example, by sutures, anchoring hooks, staples, adhesives or other means. Furthermore, certain grafts, especially tubular grafts, are combined with supporting structures known as stents which maintain the graft in an open configuration allowing fluid such as blood to pass through.

The use of attachments and supports with fabric grafts can lead to difficulties. When sutures, staples or anchoring hooks are used to attach the graft to the living animal tissue, the attachment means usually pierces the fabric of the graft, adversely affecting its tensile strength and fatigue life. The piercing may damage the filamentary members comprising the fabric and forms a failure initiation point which can lead to tearing of the fabric as it is subjected to stress within the body, with the size of the tear increasing over time. For vascular stent grafts, the piercing attachment may cause “endoleakage” of the graft and reduce the fatigue life of the graft, which must endure millions of pressure pulses as blood is pumped over the life of the patient. For a fabric mesh used to repair a hernia, the concentrated stress on the sutures at the attachment points may cause unraveling or tearing failure of the graft which is under tension in maintaining the connectivity of the abdomen walls to prevent emergence of the intestine.

Supports such as stents used in tubular stent grafts present further problems. Once implanted, there is always relative motion between the stent and the graft due to the flexibility and movement of the surrounding tissue. The relative motion leads to abrasion of the graft, since it is typically the softer material of the two components. The abrasion can, over time, cause unacceptable thinning and/or fraying of the graft, leading to endoleakage or failure of the graft.

Clearly, there is a need for fabric grafts which do not suffer from the disadvantages caused by current methods of attachment.

### SUMMARY AND OBJECTS OF THE INVENTION

The invention concerns a graft for repair of living animal tissue, the graft comprising a plurality of interlaced first filamentary members and a second filamentary member having a relatively higher tensile strength than the first filamentary members. The second filamentary member is interlaced with the first filamentary members and defines a reinforced attachment region on the graft for attachment of the graft to the living animal tissue.

Preferably, the graft comprises an elongated tube and the second filamentary member is positioned circumferentially around the tube adjacent to one end thereof. In its preferred embodiment, the graft further comprises a third filamentary member positioned circumferentially around the tube proximate to the one end and in spaced relation to the second filamentary member, the second and third filamentary members defining a space between one another comprising the attachment region, the attachment region being reinforced by the second and third filamentary members.

To improve abrasion resistance, the second and third filamentary members may have a relatively greater denier than the first filamentary members. A stent positioned at the end of the tube will engage the second and third filamentary members and, thus, be supported away from the first filamentary members by the larger denier of the second and third filamentary members. The second third filamentary members provide a sacrificial surface protecting the first filamentary members from abrasion by the stent.

The graft may also include a plurality of filamentary members positioned in the attachment region for inhibiting leaks upon attachment of the graft to the living animal tissue.

To form a tubular graft suitable for repeated cannulation (piercing), a plurality of filamentary members having a relatively higher tensile strength are interlaced in a plurality of pairs circumferentially around the tube and a plurality of pairs lengthwise along the tube thus defining a plurality of cannulation areas positioned on the tube, the cannulation areas being defined between the pairs of the filamentary members extending circumferentially around and lengthwise along the tube. Each pair of filamentary members borders one of the cannulation areas. The cannulation areas being reinforced by the filamentary members to prevent propagation of a tear in the tube from one cannulation area to another when the cannulation areas are pierced repeatedly. To prevent leakage of the cannulation areas, the tube may further comprise a plurality of filamentary members positioned within the cannulation areas on the graft for inhibiting leaks in the cannulation areas.

It is an object of the invention to provide a graft with reinforced attachment regions.

It is another object of the invention to provide a graft wherein tears which form in the attachment region do not propagate to other regions.

It is yet another object of the invention to provide a graft with an attachment region which is readily identifiable visually.

It is still another object of the invention to provide a graft which can resist abrasion in the attachment region.

It is again another object of the invention to provide a graft with areas which can be repeatedly cannulated, any tears which result from the cannulation being prevented from propagating to other cannulation regions on the graft.

These and other objects and advantages of the invention will become apparent upon consideration of the following drawings and detailed description of the preferred embodiments.

### BRIEF DESCRIPTION OF THE DRAWINGS

FIG. 1 shows a perspective view of a graft having reinforcing filamentary members according to the invention;

FIG. 2 shows a perspective view of a tubular graft having reinforcing filamentary members;

FIG. 3 is a cross-sectional view taken along line 3-3 of FIG. 2; and

FIG. 4 shows a perspective view of another embodiment of a tubular graft according to the invention.

### DETAILED DESCRIPTION OF THE PREFERRED EMBODIMENTS

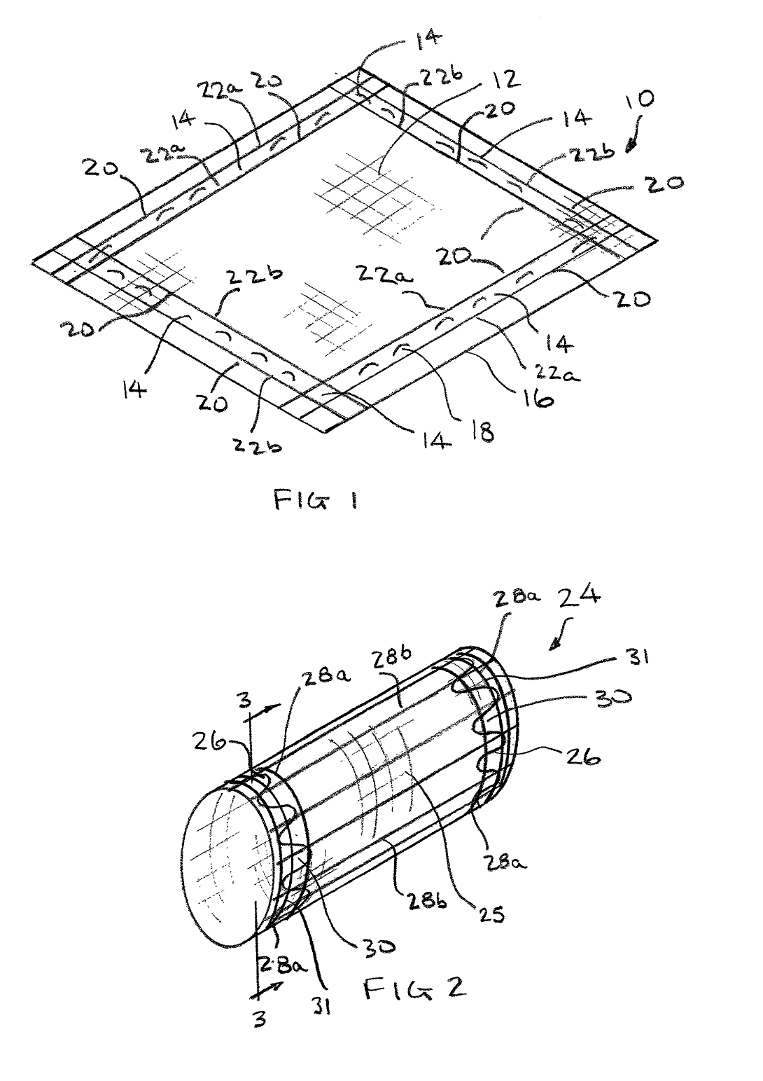

FIG. 1 shows a fabric graft 10 for repair of living animal tissue according to the invention, the graft being formed of a plurality of interlaced filamentary members 12. The term filamentary member as used herein is a generic term for a continuous strand or strands of fibers, filaments, yarns or material in a form suitable for knitting, weaving, braiding or otherwise intertwining or interlacing to form a fabric. Various forms of filamentary members include monofilaments, filaments twisted together, filaments laid together without twist, plied filaments, as well as other configurations. The filamentary members 12 used to form graft 10 preferably comprise polyester due to its compatibility with living animal tissue and success as an implant. Other feasible materials include polypropylene and nylon.

Although graft 10 could take any practical form, it is shown by way of example as a substantially flat woven patch which may be used to repair a hernia. When used for this purpose, the graft 10 is positioned over the opening in the abdominal wall and sutured to the wall at attachment regions 14 located inwardly from the perimeter 16 of the graft. When the graft is under tension, sutures 18 place concentrated point loads on the graft, which may tend to cause it to tear or unravel, either suddenly or over time. However, to prevent such a failure mode and increase the fatigue life of the graft, reinforcing filamentary members 20 are interlaced with the filamentary members 12 adjacent to attachment regions 14 to strengthen the fabric of the graft 10 in the attachment regions 14 and arrest the propagation of any tears which may initiate at the concentrated point loads caused by sutures 18.

Reinforcing filamentary members 20 have increased tensile strength relative to the filamentary members 12 comprising the majority of the graft. The increase in tensile strength may be effected in any of a number of ways. For example, the filamentary members 20 may be formed of a high strength material such as stainless steel, nitinol, titanium or some other metal wire which is compatible with living animal tissue. High strength polymers such as high tenacity polyester, polyethylene or nylon may also be used to locally reinforce the graft 10 at the attachment regions 14.

The high strength reinforcing filamentary members 20 may be incorporated into the graft in either or both the warp and fill directions for a woven or knitted graft. High strength warp filamentary members 22a are added by setting up the loom or knitting machine to feed the high strength filamentary members at certain locations in the graft. High strength fill filamentary members 22b may be inserted by using a separate shuttle which carries the high strength filamentary member and is programmed to traverse the loom at particular times during the weaving process to position the high strength filamentary members as desired. For knitted grafts, the position of the fill yarns is determined by controlling the motion in the fill direction of the knitting needles which carry the high strength filamentary members. The fill direction motion is increased when it is desired to reinforce a section of the graft, for example, the attachment regions 14.

If it is desired to effect an increase in the tensile strength of the reinforcing filamentary members 20 while using the same type of material as used for filamentary members 12, then filamentary members 20, having a higher denier and, hence, a higher tensile strength than the yarns 12, may be used. Plied filamentary members may also be used to form the reinforcing filamentary members 20. Plied filamentary members comprise a plurality of adjacent filamentary members which are woven into the graft as one and increase the strength of the graft because, like the filamentary members of relatively larger denier, they provide a localized increase in the cross sectional area over which to distribute the stress in the graft.

Larger denier or plied filamentary members may be used in either or both the warp and fill directions in the graft. When larger denier filamentary members are desired in the warp direction, the loom is set up with the larger denier filamentary members at defined locations on the warp beam. Larger denier filamentary members are included in the fill direction by using a separate shuttle to carry them, similar to the filamentary materials comprised of high strength material described above.

Plied filamentary members may be incorporated in the warp direction by coordinating the movements of adjacent heddles of the loom to be the same during weaving. Plied filamentary members are formed in the fill direction by sending the shuttle through the same shed more than once in what is known as a “dead pick” operation which lays multiple filaments adjacent to one another where normally there would be only one filamentary member. The dead pick operation is sequenced to occur when the fill filamentary members at or near the desired attachment locations 14 are being interwoven.

In addition to reinforcing the attachment regions 14, the reinforcing filamentary members may also serve as visual indicators for the proper placement of sutures, staples or other attachment means. This is accomplished by using reinforcing filamentary members 22a and 22b of a different color or colors than the yarns 12 otherwise forming the graft. Filamentary members 22a and 22b are placed far enough inwardly of perimeter 16 to ensure sufficient bight for the attachment means so that the graft does not unravel under tension. The visual indication of the reinforced region 14 between the filamentary members 22a and 22b thus avoids confusion as to where to apply the attachment means and engenders confidence that they will securely hold the graft in place.

FIG. 2 shows an example of an elongated tubular graft 24 comprising a plurality of interlaced filamentary members 25 and having stents 26 at each end to support the graft in the open configuration and force it against the inner walls of a blood vessel, for example, in the repair of an aneurysm or the preparation of an arteriovenous shunt. Tubular graft 24 has reinforcing filamentary members 28a positioned circumferentially around the graft, preferably at each end. The reinforcing filamentary members 28a have relatively higher tensile strength than the interlaced filamentary members 25 comprising the bulk of the graft 24 and define strengthened attachment regions 30 located between the filamentary members 28a. The attachment regions 30 accept sutures or other fastening means and prevent tears from initiating or propagating from the points where the sutures pierce the fabric of the graft. Stents 26 may also be secured to the graft by sutures which pierce the graft and provide further weak points. Thus, it is often advantageous to provide extended strengthened attachment regions 30 which coincide with both the region of attachment of the stent to the graft, as well as the region of attachment of the graft to a vessel.

Similar to the reinforcing filamentary members 20 used in the flat graft described above, reinforcing filamentary members 28a prevent propagation of tears and may comprise a high strength material such as stainless steel, nitinol, elgiloy, titanium or be filamentary members having a larger denier or be plied filamentary members, woven or knitted as appropriate in either or both the warp and/or fill directions. Filamentary members 28a may also have a different color or colors than filamentary members 25, thus, visually identifying the attachment regions 30 on the graft 24.

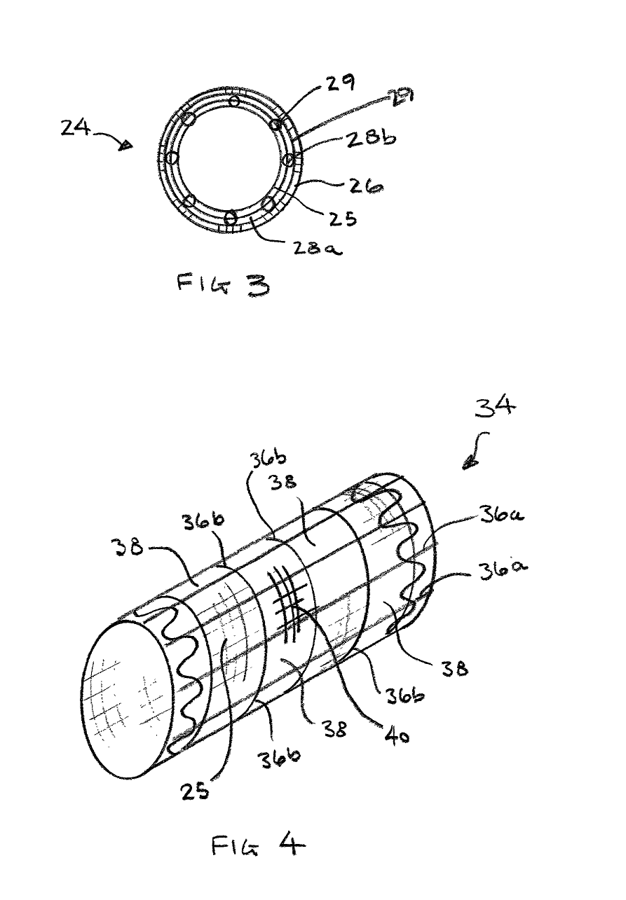

When filamentary members 28a having a larger denier are used in graft 24, the stents 26 will tend to ride on these filamentary members rather than on the filamentary members 25. The larger denier filamentary members thus act as a standoff and prevent abrasion between the stent 26 and filamentary members 25. As shown in FIG. 2 and in the cross-sectional view FIG. 3, larger denier filamentary members 28b may also be oriented in the warp direction lengthwise along the tubular graft 24. Filamentary members 28b may also be positioned between the stent 26 and the filamentary members 25 comprising the tubular graft 24. Together, the filamentary members 28a and 28b provide a sacrificial surface 29 against which the stent may rub to avoid abrading the filamentary members 25 of the graft. This feature extends the life of the graft and prevents the formation of weakened regions which may tear and leak.

An added advantage for the tubular graft 24 is obtained by the use of filamentary members 31 which serve to inhibit or prevent leaks from the graft 24. Filamentary members 31 are preferably positioned circumferentially around the graft within the attachment region 30 between the reinforcing elements 28a. Several types of filamentary members 31 may be employed to prevent leakage, as described below.

Filamentary members 31 may comprise textured filamentary members. Textured filamentary members 31 have greater bulk and will tend to block or reduce openings around the points where the sutures pierce the graft to help prevent or inhibit leakage from the graft.

Leakage around the sutures may also be reduced by forming filamentary members 31 from a heat shrinkable material such as polyester, nylon or another thermoplastic. After the stent 26 as been sutured into place, or after the graft has been sutured to a vessel, heat may be applied locally to shrink the yarns, causing them to cinch the fabric around the sutures and reduce the porosity, which may have been locally increased by the sutures or the suturing process, and thereby inhibit leakage.

Leakage may also be inhibited or prevented by making filamentary members 31 from elastic material. Elastic filamentary members 31 will deflect when the fabric which they comprise is pierced but will attempt to return to their original position due to their elastic biasing. The elastic filamentary members will thus tend to snug up against the sutures and seal the hole in the fabric caused by the suturing process.

FIG. 4 shows a substantially impermeable tubular graft 34 comprising a plurality of interlaced filamentary members 25 and a plurality of pairs of interlaced filamentary members 36a and 36b. Filamentary members 36a and 36b are arranged both lengthwise along graft 34 and circumferentially about the graft respectively and have relatively higher tensile strength than filamentary members 25. The pairs of filamentary members 36a and 36b define a plurality of cannulation areas 38 along graft 34. Such a graft is useful to form a prosthetic arteriovenous shunt for patients requiring hemodialysis due to renal failure. In hemodialysis, the patient's blood is passed through a filter which removes unwanted substances from the blood. Access to the circulation is required to conduct the blood to the filter. This is accomplished by an arteriovenous shunt or fistula which connects an artery to a nearby vein. The shunt or fistula is cannulated (pierced) with needles of the proper gauge which are connected to the filter and allow the blood to flow to it at a sufficient rate for proper dialysis. Whether formed from a natural vein or a prosthetic graft, the repeated cannulation of the shunt causes damage to it over time. The use of graft 34 as a prosthetic arteriovenous shunt, the graft having cannulation areas 38 reinforced by filamentary members 36a and 36b, should provide a longer lasting shunt which can withstand the repeated cannulation better than a shunt made from a natural vein or a synthetic graft due to the presence of the higher tensile strength filamentary members 36a and 36b which will help prevent tears from occurring and propagating into other cannulation areas when the needles pierce the fabric. The higher tensile strength filamentary members 36a and 36b may be formed as described above, by using materials having inherently greater strength, filaments having larger denier or by plying multiple filaments together.

Graft 34 may further comprise filamentary members 40 adapted to inhibit or prevent leakage from the cannulation areas 38. Filamentary members 40 are interlaced with filamentary members 25 and located within the cannulation areas, either or both lengthwise and circumferentially around the graft. Leak inhibiting filamentary members 40 may comprise textured filamentary members and/or elastic filamentary members as described previously. Such leak inhibiting filamentary members will inhibit leakage from the cannulation areas during and after cannulation by the various mechanisms described above.

The use of leak and tear resistant graft fabrics according to the invention promises to improve the performance of grafts in any application when implanted in a living body by providing more robust devices which have greater strength where required, greater resistence to damage, increased fatigue and wear life and improved surface characteristics as required to prevent or inhibit leakage at points of attachment or cannulation.

What is claimed is:

1. In a graft for repair of living animal tissue, said graft comprising a plurality of interlaced first filamentary members, a second filamentary member having a relatively higher tensile strength than said first filamentary members, said second filamentary member being interlaced with said first filamentary members and defining a reinforced attachment region on said graft for attachment of said graft to said living animal tissue.

2. A graft according to claim 1, wherein said second filamentary member comprises a material of a composition characterized by a higher tensile strength relatively to material comprising said first filamentary members.

3. A graft according to claim 2, wherein said material comprising said second filamentary member is selected from the group consisting of nitinol, stainless steel and titanium.

4. A graft according to claim 3, wherein said material comprising said first filamentary members is selected from the group consisting of polyester, polypropylene and nylon.

5. A graft according to claim 1, wherein said second filamentary member comprises a plurality of plied filaments.

6. A graft according to claim 5, wherein each filament of said plurality of plied filaments has substantially the same denier and comprises substantially the same material as said first filamentary members comprising said graft.

7. A graft according to claim 1, wherein said second filamentary member has a relatively greater denier than said first filamentary members.

8. A graft according to claim 1, wherein said graft comprises an elongated tube.

9. A graft according to claim 8, wherein said second filamentary member is positioned circumferentially around said tube adjacent to one end thereof.

10. A graft according to claim 9, further comprising a third filamentary member positioned circumferentially around said tube proximate to said one end and in spaced relation to said second filamentary member, said second and third filamentary members defining a space between one another comprising said attachment region, said attachment region being reinforced by said second and third filamentary members.

11. A graft according to claim 10, wherein said second and third filamentary members have a color different from said first filamentary members comprising said graft, said second and third filamentary members, thereby visually identifying said attachment region therebetween.

12. A graft according to claim 11, wherein said first, second and third filamentary members are interlaced by weaving.

13. A graft according to claim 10, further comprising a stent extending circumferentially around said tube and attached thereto at said one end thereby supporting said tube radially outwardly, said stent engaging said second and third filamentary members.

14. A graft according to claim 13, wherein said second and third filamentary members have a relatively greater denier than said first filamentary members, said stent being supported away from said first filamentary members by said second and third filamentary members, said second third filamentary members providing a sacrificial surface protecting said first filamentary members from abrasion by said stent.

15. A graft according to claim 14, further comprising a plurality of longitudinal filamentary members oriented substantially lengthwise along said tube and positioned circumferentially there around, said longitudinal filamentary members being interlaced with and having a relatively greater denier than said first filamentary members and intersecting said second and third filamentary members at said one end thereby further supporting said stent away from said first filamentary members.

16. In a graft for repair of living animal tissue, said graft comprising a plurality of interlaced first filamentary members, a plurality of second filamentary members interlaced with said first filamentary members and positioned in an attachment region on said graft attachable to said living animal tissue, said second filamentary members for inhibiting leaks upon attachment of said graft to said living animal tissue.

17. A graft according to claim 16, wherein said graft comprises an elongated tube, said second filamentary members being positioned circumferentially around said tube at one end thereof.

18. A graft according to claim 17, wherein said first and second filamentary members are interlaced by weaving, said second filamentary members comprising textured filamentary members having increased bulk adapted to overlie and block openings in said attachment region resulting from piercing of said attachment region during attachment of said graft to said living animal tissue.

19. A graft according to claim 17, wherein said first and second filamentary members are interlaced by weaving, said second filamentary members comprising a heat-shrinkable material which shrinks upon heating to cinch said one end of said tube and close openings in said attachment region resulting from piercing of said attachment region during attachment of said graft to said living animal tissue.

20. A graft according to claim 19, wherein said heat-shrinkable material comprises a thermoplastic.

21. A graft according to claim 17, wherein said first and second filamentary members are interlaced by weaving, said second filamentary members comprising an elastic material which deflects elastically from a nominal position to form a temporary opening when pierced during attachment of said graft to said living animal tissue, said second filamentary members returning substantially to said nominal position and thereby closing said opening in said attachment region resulting from said piercing of said attachment region.

22. A graft compatible with living animal tissue, said graft comprising:
an elongated tube formed of a plurality of interlaced first filamentary members, said tube being relatively impermeable and useable as a fluid conduit;
a plurality of second filamentary members having a relatively higher tensile strength than said first filamentary members, said second filamentary members being interlaced with said first filamentary members in a plurality of pairs circumferentially around said tube and a plurality of pairs lengthwise along said tube; and
a plurality of cannulation areas positioned on said tube, said cannulation areas being defined between said pairs of said filamentary members extending circumferentially around and lengthwise along said tube, each said pair of filamentary members bordering one of said cannulation areas, said cannulation areas being reinforced by said filamentary members to prevent propagation of a tear in said tube from one cannulation area to another.

23. A graft according to claim 22, wherein said second filamentary members comprise a material having a higher tensile strength than said first filamentary members.

24. A graft according to claim 22, wherein said second filamentary members each comprise a plurality of plied filaments.

25. A graft according to claim 24, wherein each of said plied filaments has substantially the same denier and comprises substantially the same material as said first filamentary members.

26. A graft according to claim 22, wherein said second filamentary members have a relatively greater denier than said first filamentary members.

27. A graft according to claim 26, wherein said second filamentary members comprise substantially the same material as said first filamentary members.

28. A graft according to claim 22, further comprising a plurality of third filamentary members interlaced with said first filamentary members and positioned within said cannulation areas on said graft, said third filamentary members for inhibiting leaks in said cannulation areas.

29. A graft according to claim 28, wherein said third filamentary members comprise textured filamentary members having increased bulk adapted to overlie and block openings in said cannulation areas resulting from piercing of said areas during cannulation.

30. A graft according to claim 28, wherein said third filamentary members comprise an elastic material, said third filamentary members deflecting elastically from a nominal position to form a temporary opening when pierced during cannulation of said cannulation areas, said third filamentary members returning substantially to said nominal position after said cannulation and thereby closing said opening in said cannulation area resulting from said piercing.
